(19) United States
(12) Patent Application Publication
(10) Pub. No.: US 2013/0072869 A1
(43) Pub. Date: Mar. 21, 2013

(54) Intra-Gastric Timed-Release Drug Delivery System

(71) Applicants: Protagoras N. Cutchis, Highland, MD (US); Steven M. Babin, Greenbelt, MD (US)

(72) Inventors: Protagoras N. Cutchis, Highland, MD (US); Steven M. Babin, Greenbelt, MD (US)

(73) Assignee: THE JOHNS HOPKINS UNIVERSITY, Baltimore, MD (US)

(21) Appl. No.: 13/599,080

(22) Filed: Aug. 30, 2012

Related U.S. Application Data
(60) Provisional application No. 61/535,151, filed on Sep. 15, 2011.

(51) Int. Cl. A61M 25/10

(52) U.S. Cl. 604/103.02

(57) ABSTRACT

A timed-release drug delivery assembly includes an expandable structure and drug microparticles. The expandable structure is defined by host material that causes expansion of the expandable structure. The expandable structure has a non-expanded state prior to exposure to a gastric environment during which a size of the expandable structure enables entry of the expandable structure into a stomach of a patient, and an expanded state after exposure to the gastric environment during which the size and shape of the expandable structure prevents exit of the expandable structure from the stomach. The drug microparticles are held at least in part by the host material to enable release of the drug microparticles into the gastric environment over a predefined period of time. The expandable structure may include, in the expanded state, a substantially hollow framework to avoid blocking a pyloric valve of the stomach.

### CROSS-REFERENCE TO RELATED APPLICATIONS

This application claims priority to and the benefit of co-pending U.S. Provisional Application No. 61/535,151 filed on Sep. 15, 2011, the entire contents of which are hereby incorporated herein by reference.

### STATEMENT OF GOVERNMENTAL INTEREST

This invention was made with U.S. Government support under contract number 04-D-4008. The U.S. Government has certain rights in the invention.

### TECHNICAL FIELD

Example embodiments generally relate to timed-release delivery of drugs and, more particularly, relate to a timed-release delivery system that includes an expandable structure that expands in the stomach to keep the structure in the stomach for a predetermined period of time without blocking stomach function, but while enabling long term delivery of the drug to the patient or host.

### BACKGROUND

One of the major factors associated with achieving positive outcomes in the treatment of patients is medication adherence in relation to a particular treatment regimen. Generally speaking, the term “regimen” may be descriptive of a treatment plan defined for a particular patient. The regimen for the patient may include a combination of drugs, their doses and administration techniques along with a schedule for how often the drugs are to be administered. If the patient takes the proper combination of drugs via the proper techniques and at the prescribed schedule, the treatment regimen has a higher likelihood of success.

Failures in medication adherence can be very costly to the health care industry, to the public at large, and to the patient. Failure to take medications in accordance with instructions may obviously cause wastage with respect to medications not taken or not taken in a manner that is likely to be effective. However, the costs of failures in medication adherence also extends to cover the costs of complications that arise from failure to adhere and/or the costs of recurrence or lingering of illness and the subsequent treatments that may continue or even expand. In some cases, failures in medication adherence can even cause treatment resistant strains of certain pathogens to be created, which can be a threat to public health.

The healthcare industry has been making increased efforts to use education, incentives and even technically implemented reminder systems to attempt to improve medication adherence. However, even these efforts cannot ensure that medication adherence is practiced in all instances. Moreover, some patients may be in situations that make medication adherence extremely difficult or even a secondary consideration for survival. For example, a warfighter that is wounded in a remote area and needs to take an antibiotic over a period of days to weeks may find it extremely difficult to keep, much less take, a series of pills over many days. Additionally, some patients that have track records of poor adherence may not be properly treatable over a period of time unless they can be closely supervised, and such supervision may not be possible or cost effective.

### BRIEF SUMMARY

Accordingly, some example embodiments may enable the provision of a timed-release drug delivery system that can be taken orally at one time and still provide for delivery of the drug over a prolonged period of time (e.g., days to weeks). Thus, in some cases, particularly where the treatment regimen requires only a single oral administration, but the drug can be released over the prolonged period of time, medication adherence can be assured. As such, the likelihood of positive outcomes may be increased, even in environments (e.g., battlefield environments) where medication adherence could otherwise be very challenging to achieve.

In one example embodiment, a timed-release drug delivery assembly is provided. The timed-release drug delivery assembly may include an expandable structure and drug microparticles. The expandable structure may be defined by host material that causes expansion of the expandable structure responsive to exposure to a gastric environment. The expandable structure may have a non-expanded state prior to exposure to the gastric environment during which a size of the expandable structure enables entry of the expandable structure into a stomach of a patient. The expandable structure may also have an expanded state after exposure to the gastric environment during which the size and shape of the expandable structure prevents exit of the expandable structure from the stomach. The drug microparticles may be held at least in part by the host material to enable release of the drug microparticles into the gastric environment over a predefined period of time. The expandable structure may include, in the expanded state, a substantially hollow framework to avoid blocking a pyloric valve of the stomach and thus allow passage of food and drink.

In another example embodiment, a timed-release drug delivery assembly is provided. The timed-release drug delivery assembly may include an expandable structure and drug microparticles. The expandable structure may be defined by host material. The expandable structure may have a non-expanded state prior to exposure to a gastric environment during which a size of the expandable structure enables entry of the expandable structure into a stomach of a patient, and the expandable structure may have an expanded state after exposure to the gastric environment during which the size and shape of the expandable structure prevents exit of the expandable structure from the stomach. The drug microparticles may be held at least in part by the host material to enable release of the drug microparticles into the gastric environment over a predefined period of time. The expandable structure may include, in the expanded state, a substantially hollow framework to avoid blocking a pyloric valve of the stomach and thus allow passage of food and drink. The change in state from the non-expanded state to the expanded state may be caused by exposure of the host material to a low pH of the gastric environment to cause expansion of the host material.

### BRIEF DESCRIPTION OF THE SEVERAL VIEWS OF THE DRAWING(S)

Having thus described several exemplary embodiments of the invention in general terms, reference will now be made to the accompanying drawings, which are not necessarily drawn to scale, and wherein:

FIG. 1 illustrates an example of a timed-release drug delivery assembly of an example embodiment entering into a gastric environment;

FIG. 2 illustrates a cross sectional view of the timed-release drug delivery assembly according to an example embodiment;

FIG. 3 illustrates a conceptual view of one form that an expanded structure may take in an expanded state within the stomach of a patient according to an example embodiment;

FIG. 4 illustrates a conceptual view of one form that an expanded structure may take in an expanded state within the stomach of a patient according to an example embodiment;

FIG. 5 illustrates a conceptual view of one form that an expanded structure may take in an expanded state within the stomach of a patient according to an example embodiment sphere;

FIG. 6 illustrates a cross section view of a portion of the expandable structure according to an example embodiment; and

FIG. 7 illustrates a cross section view of a portion of an alternative arrangement for the expandable structure according to an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout.

As indicated above, improvements in medication adherence may be provided by enabling a single administration of a pill or other drug dispenser to provide a time-released dose to the patient instead of requiring the patient to take multiple administrations of the drug over a predefined period of time. Such a time-released dose provision mechanism may not only improve adherence, but may be a more convenient or practical way to deliver medication to patients in some environments and under certain challenging conditions.

One way to provide a time-released dose to a patient may be to provide a pill that can be taken orally and expand in the stomach to remain there for a period of time. However, providing a pill that swells in size to prevent passage out of the stomach may necessarily block the pyloric valve at the exit of the stomach. Such blockage may prevent passage of food and drink from the stomach and lead to serious discomfort. Accordingly, any attempt to provide such a swelling pill may be required to only enable the pill to swell for a relatively limited period of time. Thus, such a solution may not be practical for drugs that need several days of doses in order to complete the regimen.

Some example embodiments may therefore enable the provision of a time-released delivery system that can stay in the stomach for extended periods of time without blocking or inhibiting stomach function. In this regard, for example, some embodiments may provide a drug dosage delivery system in the form of an expandable structure that can expand after ingestion, but not block the pyloric valve. To achieve such a structure, the expandable structure may take the shape of a hollow framed structure so that food and drink can pass through the hollow framed structure.

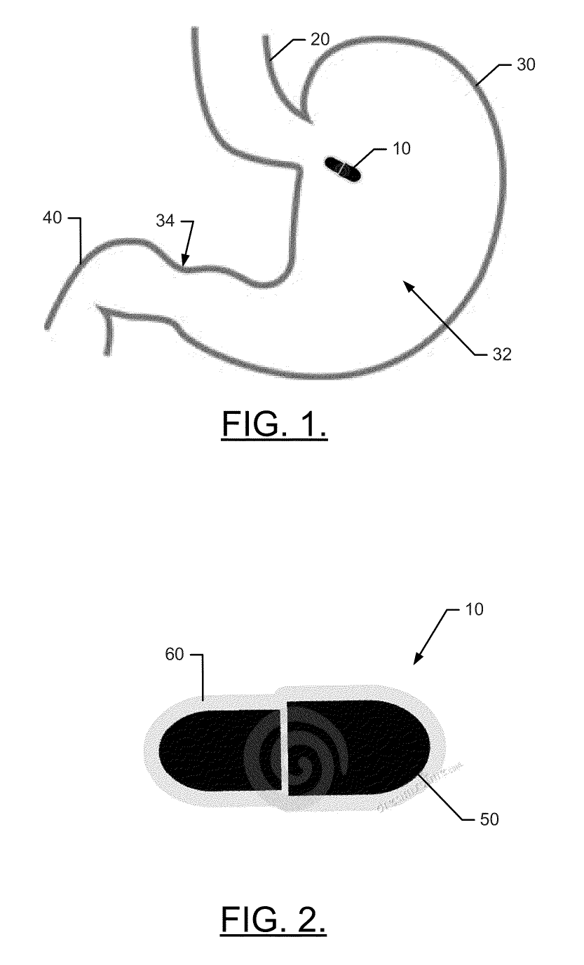

FIG. 1 illustrates an example of a timed-release drug delivery assembly 10 of an example embodiment entering into a gastric environment. The timed-release drug delivery assembly 10, or drug dispenser, may take the form of a pill that is ingestible into the mouth of a patient. It should be appreciated that, although the term “patient” is used herein, example embodiments may not always be employed purely in a medical treatment environment. Thus, medicinal drugs, drugs that provide nutrition supplements, or any other drug could be administered using example embodiments so that the term “patient” should be understood more broadly to include any host that might require or desire administration of a drug via the time-released mechanism described herein.

After the timed-release drug delivery assembly 10 is swallowed by the patient, the timed-release drug delivery assembly 10 may transit down the esophagus 20 of the patient to the stomach 30. Within the stomach 30, a gastric environment 32 may be provided having a characteristically low pH for processing substances that are provided therein. The substances may be broken down, at least in part, by the gastric environment 32 before exiting the stomach 30 via the pyloric valve 34, which may lead to the duodenum 40 at the beginning of the small intestine.

FIG. 2 illustrates a cross sectional view of the timed-release drug delivery assembly 10 according to an example embodiment. As shown in FIG. 2, the timed-release drug delivery assembly 10 may enclose an expandable structure 50 within a capsule 60. The capsule 60 may be made of a dissolvable material that, responsive to swallowing and entry into the stomach 20 dissolves in the gastric environment 32. In some embodiments, for example, the capsule 60 may be made from natural cellulosic gels. When the capsule 60 dissolves, the expandable structure 50 may be exposed and enabled to expand in the gastric environment 32. It should be appreciated, however, that in some embodiments, the expandable structure 50 may not necessarily be provided within the capsule 60. Instead, the expandable structure 50 may be provided in a pill form with or without a layer of dissolvable material on the outside thereof (e.g., a dissolvable coat). In any case, however, the expandable structure 50 may be provided with or otherwise carry drug microparticles that are released in the stomach 30 over time as described in greater detail below.

In some cases, the expandable structure 50 may be defined by host material that causes expansion of the expandable structure 50 responsive to exposure to a gastric environment 32. As such, in some embodiments, the expandable structure 50 may have a non-expanded state prior to exposure to the gastric environment 32 during which a size and shape of the expandable structure 50 enables entry of the expandable structure into a stomach 30 of a patient, and the expandable structure having an expanded state after exposure to the gastric environment 32. In the expanded state, the size of the expandable structure may be increased relative to the size of the expandable structure in the non-expanded state. In some cases, the shape may also change. However, the shape may be relatively consistent in each state, except to the extent that the size increase may change the dimensions of some or all of the aspects of the shape. In any case, the increase in size of the expandable structure 50 in the expanded state may prevent the exit of the expandable structure 50 from the stomach 30. Moreover, the shape of the expandable structure 50 may be such that it facilitates keeping the expandable structure 50 within the stomach 30 in the expanded state, while not impacting stomach function in any negative way. In some embodiments, the expandable structure 50 may be made of host material that is activated by the low pH of the gastric environment 32. However, in other alternative embodiments, the host material of the expandable structure 50 may simply be activated to expand responsive to release from the compression provided by the capsule 60. In other words, the expandable structure 50 may be compressed within the capsule 60 and, responsive to the capsule 60 dissolving, the expandable structure 50 may no longer be prevented from expanding and may therefore expand. In still other alternative embodiments, the expandable structure 50 may include host material that releases gas as it dissolves in the gastric environment 32. For example, sodium bicarbonate and citric acid may be combined to release carbon dioxide when the host material is exposed to the gastric environment 32. The gas released may then be used to fill the expandable structure. In still other embodiments, the host material may be dehydrated in the non-expanded state and may expand responsive to hydration in the gastric environment 32. As such, for example, the host material may include dried fibers (e.g., psyllium) that may swell when liquid is absorbed by the fibers.

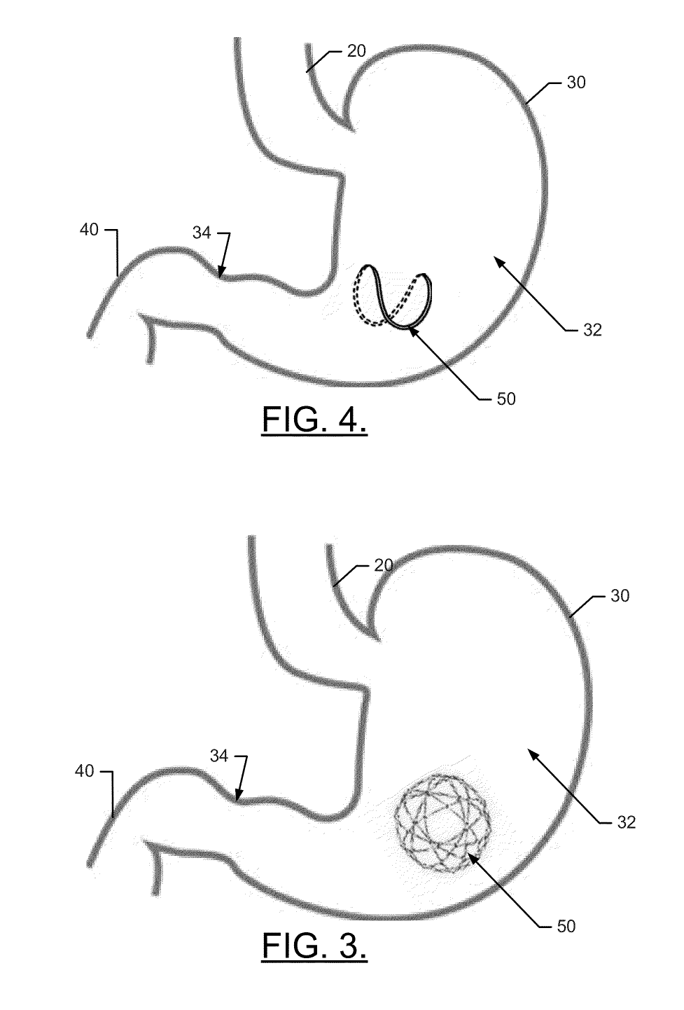

Regardless of the particular mode of activation and the cause for expansion, after the expandable structure 50 is altered within the gastric environment 32 to be in the expanded state, the expandable structure 50 may be prevented from passing the pyloric valve 40 for as long as the expandable structure 50 remains intact in its expanded state. FIGS. 3-5 illustrate some examples of shapes that may be employed in connection with expandable structures in their respective expanded states according to some alternative example embodiments. However, it should be appreciated that expandable structures of any shape may be utilized as long as they do not block the passage of food and drink out of the pyloric valve 34, while preventing such structures from passing through the pyloric valve 34 themselves.

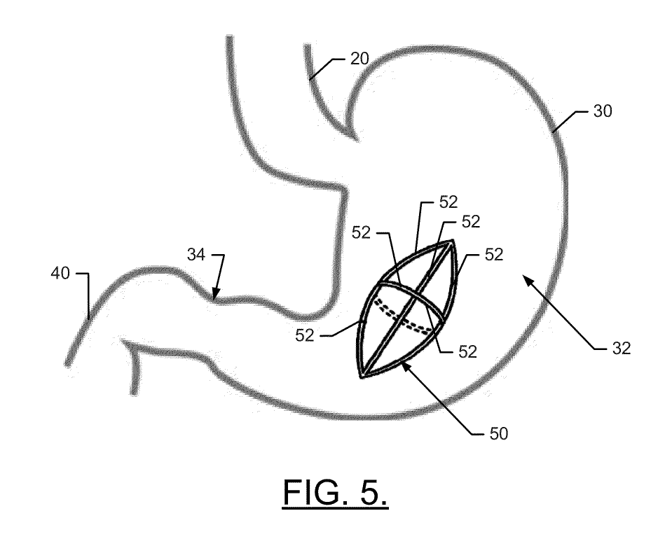

In an example embodiment, the expanded structure 50 may take the form of a substantially hollow framework in the expanded state in order to avoid blocking the pyloric valve 34 of the stomach 30 and thus to allow passage of food and drink out of the stomach 30. This hollow framework structure may enable the expanded structure 50 to stay within the stomach 30 for several days, and in some cases longer than a week, since there may not be any noticeable effect on stomach function. In some embodiments, the expanded structure 50 may take the form of a toroid in the expanded state so that food and drink can pass through the hollow center of the toroid. As an alternative, and as shown in FIG. 3, in one example embodiment, the expanded structure 50 may take the form of an expandable spherical framework (e.g., a Hoberman sphere) that allows food and drink to pass through the hollow center. Meanwhile, in the example of FIG. 4, a toroid structure may be formed in a bent arrangement that approximates the shape of the laces of a baseball by forming a closed loop, topological circle that is elongated and then bent about a transverse centerline of the elongated structure. In the example of FIG. 5, a plurality of elongated members 52 are connected to form a hollow framework by connecting ends of the elongated members to corresponding ends of other members to form the shape of a football with the inside of the shape again being hollow so that food and drink may pass there through.

Regardless of the particular shape of the expandable structure 50, the substantially hollow framework of the expandable structure 50 in the expanded state may prevent interference with stomach function. Of note, even solid food that is provided into the stomach 30 is converted to a semi-liquid substance referred to as chyme. Thus, after food is processed in the stomach 30 and moved toward the exit of the stomach (e.g., at the pyloric valve 34), the contents of the stomach 30 are in a form that may egress via the pyloric valve 34, through the substantially hollow framework of the expandable structure 50 in the expanded state without being obstructed or caught up in the expandable structure 50. In other words, the chyme may flow through the expandable structure 50 in its expanded state without being blocked by the expandable structure 50.

Each of the shapes formed in FIGS. 3-5 may have a size sufficient to prevent passage of the expandable structure 50 from the stomach 30 for a predefined period. In this regard, the host material forming the expandable structure 50 may be selected and/or produced such that the host material degrades, is digested, or is otherwise processed to the point where its integrity will fail after the predetermined period of time (e.g., over a period of days to weeks). When the integrity of the expandable structure 50 fails, the remaining materials will be passed through the intestinal tract as waste.

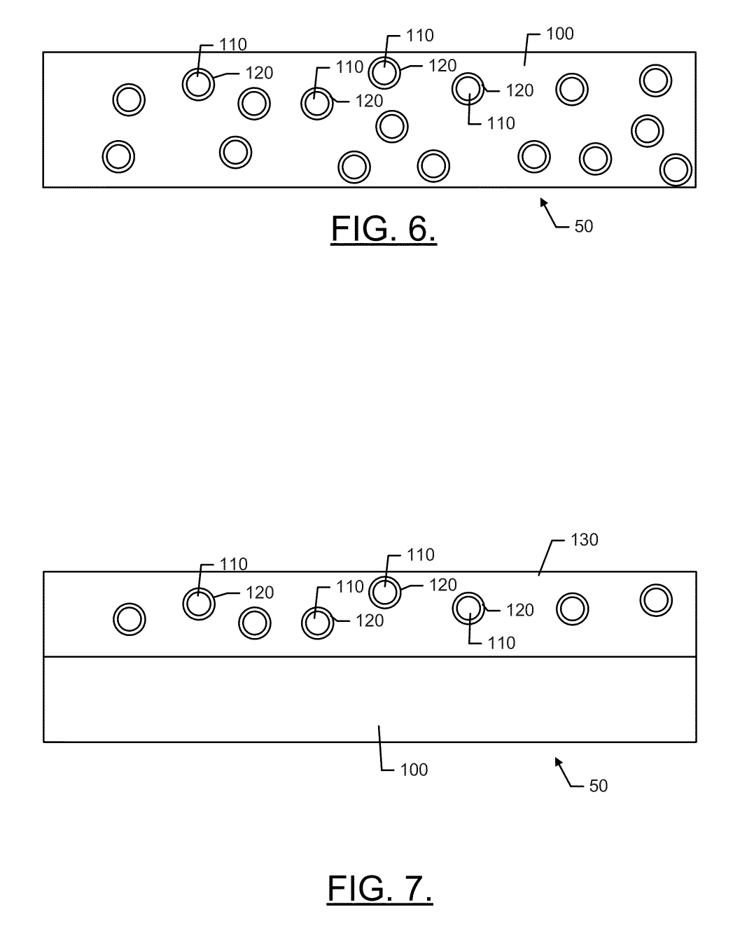

Meanwhile, for the period of time that the expandable structure 50 remains in the stomach 30, drug microparticles in the expandable structure 50 may be delivered to the body via absorption in the stomach 30 or the duodenum 40. In some embodiments, the drug microparticles may be disposed in a matrix throughout the host material of the expandable structure 50. In this regard, the example of FIG. 6 shows a cross section view of a portion of the expandable structure according to an example embodiment. As an example, FIG. 6 may be a cross sectional view taken along a longitudinal axis of one of the elongate members 52 of FIG. 5. However, it should be appreciated that the cross section view of FIG. 6 may also represent any other portion of the expandable structure 50, and that alternative structures may also fall within the scope of example embodiments.

In this example, host material 100 is provided with drug particles 110 interspersed throughout the host material 100. Although not required, the drug particles 110 of this example further include a coating material 120 disposed around a periphery of each respective one of the drug particles 110. When the coating material 120 is used, the drug particles 110 may be released into the stomach 30 as the host material 100 degrades over time within the gastric environment 32. As each drug particle 110 is released, the corresponding dose of the drug may, for example, be delivered to the patent either through absorption in the stomach 30 (e.g., if the coating material 120 is selected such that it dissolves in the stomach 30), or in the duodenum 34 (e.g., if the coating material 120 is selected such that it dissolves in the pancreatic enzymes of the duodenum 34). For example, the coating material 120 may be amylose, a starch that is digested by pancreatic amylase to cause the coating material 120 to dissolve in the duodenum 34 or a collagen material to cause the coating material 120 to dissolve in the stomach 30.

In some embodiments, the density of the drug particles 110 may be altered within the host material 100 so that an even distribution is obtained. Alternatively, the density may be arranged to provide dose delivery rates that are uneven if such a regimen is preferred for any reason. In altering the density, it should be appreciated that if the host material 100 dissolves, the drug particles 110 proximate to the outer periphery of the host material 100 will be released first. As the periphery of the host material 100 recedes due to continued dissolving of the host material, drug particles 110 closer to the center of the host material 100 will also be released.

As an alternative to the example of FIG. 6, FIG. 7 illustrates an example in which the host material 100 itself does not release the drug particles 110. In the example of FIG. 7, part or all of the host material 100 may be coated with coating layer 130. The drug particles 110 may be distributed within the coating layer 130 (with or without coating material 120 on the drug particles 110). In this example, the coating layer 130 may be dissolved over time and release the drug particles 110 as described above. In such an example, the density of the drug particles 110 may be controlled as described above. The coating layer 130 may be made from processed collagen that may be designed to dissolve in the gastric environment 32 over a period of days. In some cases, multiple layers of coating (e.g., multiple coating layers) may be provided and each layer may be provided with corresponding different drug particles to provide different courses of drug therapy while the expandable structure 50 is within the stomach 30.

By employing a dissolvable material such as collagen as the host material 100 or the coating layer 130, stay times in the stomach 30 for the expandable structure can be achieved in excess of three days. Moreover, in some embodiments, stay times of greater than seven to ten days may be achieved. During the stay time period, a continuous or otherwise controllable dose of drug particles may be delivered to the patient. Thus, for example, if a warfighter is injured and needs to take medication to fight infection, one administration of the timed-release drug delivery assembly 10 may be taken. However, the timed-release drug delivery assembly 10 may expand into the expanded state of the expandable structure 50 and stay in the stomach 30 long enough to maintain antibiotic levels in the blood stream of the patient sufficient to eradicate infectious agents. Meanwhile, normal stomach function may not be interrupted and the expandable structure 50 itself will also eventually degrade and pass through the stomach 30 and intestinal tract to be processed normally as waste.

Example embodiments may therefore provide for a mechanism by which to introduce a drug delivery device or dispenser (e.g., the timed-release drug delivery assembly 10) into the body of a patient orally in one administration. The single administration may, however, be retained in the body (e.g., in the stomach) over a predetermined period of time to deliver a dose of one or more selected drugs into the bloodstream of the patient responsive to timed-release of the drug particles while the timed-release drug delivery assembly 10 remains in the stomach 30. The timed-release drug delivery assembly 10 employs a structure that expands in the gastric environment 32 in order to maintain the structure in the stomach 30. However, although the timed-release drug delivery assembly 10 includes the expandable structure 50, such structure is designed to be a hollow framework after it expands to its expanded state so that stomach function is not impacted. Thus, the expanded structure 50 can remain in the stomach 30 for several days without causing discomfort or other complications for the patient.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

What is claimed is:

1. A timed-release drug delivery assembly comprising:
an expandable structure defined by host material that causes expansion of the expandable structure responsive to exposure to a gastric environment, the expandable structure having a non-expanded state prior to exposure to the gastric environment during which a size of the expandable structure enables entry of the expandable structure into a stomach of a patient, and the expandable structure having an expanded state after exposure to the gastric environment during which the size and shape of the expandable structure prevents exit of the expandable structure from the stomach; and
drug microparticles held at least in part by the host material to enable release of the drug microparticles into the gastric environment over a predefined period of time,
wherein the expandable structure comprises, in the expanded state, a substantially hollow framework to avoid blocking a pyloric valve of the stomach and thus allow passage of food and drink.

2. The assembly of claim 1, wherein the substantially hollow framework has a diameter that is larger than a diameter of the pyloric valve.

3. The assembly of claim 1, wherein the host material expands in size responsive to exposure to liquid in the gastric environment.

4. The assembly of claim 3, wherein a change in state from the non-expanded state to the expanded state is caused by hydration of dehydrated host material to cause expansion of the host material responsive to liquid absorption.

5. The assembly of claim 1, wherein the host material comprises a gas releasing material that releases gas to form the expandable structure in the expanded state in the gastric environment.

6. The assembly of claim 1, wherein the gas released fills the expandable structure to form the expandable structure in the expanded state.

7. The assembly of claim 1, further comprising a capsule that is ingestible by the patient, the capsule enclosing the expandable structure and dissolving in the stomach to expose the expandable structure to the gastric environment.

8. The assembly of claim 7, wherein the expandable structure is compressed within the capsule in the non-expanded state and decompresses to expand to the non-expanded state responsive to the capsule dissolving.

9. The assembly of claim 1, wherein the host material is configured to dissolve over a period of greater than three days.

10. The assembly of claim 9, wherein the host material comprises processed collagen.

11. The assembly of claim 1, further comprising a dissolvable coating material to initially retain the drug microparticles proximate to the host material and release the drug microparticles into the gastric environment responsive to the dissolvable coating material being dissolved by the gastric environment.

12. The assembly of claim 11, wherein the drug microparticles are disposed at different depths within the dissolvable coating material such that, as the dissolvable coating material dissolves over time, distributed release of the drug particles into the gastric environment is achieved.

13. The assembly of claim 12, wherein the dissolvable coating material is configured to dissolve over a period of greater than three days.

14. The assembly of claim 13, wherein the dissolvable coating material comprises processed collagen.

15. The assembly of claim 1, wherein the drug microparticles further comprise a particle coating disposed thereon, the particle coating comprising a substance that is dissolvable in a duodenum of the patient after release of the drug microparticles into the gastric environment.

16. The assembly of claim 15, wherein the substance forming the particle coating comprises amylose.

17. The assembly of claim 1, wherein the substantially hollow framework comprises a toroid shape.

18. The assembly of claim 1, wherein the substantially hollow framework comprises a plurality of elongate members that are arranged to attach to adjacent elongate members at respective ends thereof.

19. The assembly of claim 1, wherein the drug microparticles are distributed in a matrix throughout at least a portion of the host material.

20. A timed-release drug delivery assembly comprising:
an expandable structure defined by host material, the expandable structure having a non-expanded state prior to exposure to a gastric environment during which a size of the expandable structure enables entry of the expandable structure into a stomach of a patient, and the expandable structure having an expanded state after exposure to the gastric environment during which the size and shape of the expandable structure prevents exit of the expandable structure from the stomach; and
drug microparticles held at least in part by the host material to enable release of the drug microparticles into the gastric environment over a predefined period of time,
wherein the expandable structure comprises, in the expanded state, a substantially hollow framework to avoid blocking a pyloric valve of the stomach and thus allow passage of food and drink, and
wherein a change in state from the non-expanded state to the expanded state is caused by exposure of the host material to a low pH of the gastric environment to cause expansion of the host material.
